(19) United States
(12) Patent Application Publication
(10) Pub. No.: US 2011/0264377 A1
(43) Pub. Date: Oct. 27, 2011

(54) METHOD AND SYSTEM FOR ANALYSING DATA SEQUENCES

(76) Inventor: John Gerald Cleary, Hamilton (NZ)

(21) Appl. No.: 13/129,329

(22) PCT Filed: Nov. 13, 2009
(86) PCT No.: PCT/NZ2009/000245

§ 371 Date: Jul. 14, 2011

(30) Foreign Application Priority Data
Nov. 14, 2008 (NZ) 572847

(51) Int. Cl. G01N 33/48; G06F 19/10

(52) U.S. Cl. 702/19

(57) ABSTRACT

A sequencing system and method of generating index keys for one or more data sequence based on masked values of reads from a sample data sequence and/or one or more template data sequence. Each index key value may be based upon a concatenated form of each extracted value, although other transformations may be employed. A number of different masks may be applied to the data sequence at a number of locations. At least some of the masks may include indels and/or substitutions. The masks may be manually or computer generated. The data sequence may be one or more reference templates and/or one or more sample sequences, such as DNA or RNA sequences. Sample data may be stored in the one or more index by correlating masked values of reads with index key values and storing an identifier for each read in association with a corresponding index key value. Sample data sequences may be evaluated by comparing sample sequence and template sequences having the same index key value and determining scores for the reads based on the comparison and associating the scores with the reads. Reads may be rejected based upon the comparison. A read may be rejected if there is more than one position at which it has a best score. A read may be rejected if its score falls below a threshold score level.

### FIELD

The present invention relates to a method and system for analysing data sequences based on the use of index values. The method is particularly suitable for rapidly matching sequences of nucleotides (RNA or DNA) extracted from individual organisms but is also applicable to the analysis of other large complex data sequences.

### BACKGROUND

Recently there has been an explosion of data on genomic sequences from many organisms including humans, bacteria and many other species. This data may be taken from the organism's DNA or RNA. First the DNA or RNA is extracted from the organism, and is prepared chemically. Then the sequencing machines produce short sequences, called reads, from approximately 15 nucleotides up to hundreds or thousands of nucleotides. Each of these reads corresponds to a part of the DNA or RNA extracted from the organism.

The reads occur randomly throughout the DNA or RNA. In order to extract statistically meaningful information about the particular organisms DNA or RNA it is necessary to have many reads. This is typically measured by the coverage of a set of reads, which means the number of times on average each nucleotide in the DNA or RNA would be covered by different reads. A typical example, with a set of reads of length 30 and coverage of 15 on the human genome, requires some 1.7 billion reads.

Another characteristic of these reads is that there are often small errors in them. These errors can be either a substitution where one nucleotide is erroneously read as a different one or an indel where one or more nucleotides are inserted or deleted. In a typical example it might be desired to allow for up to 4 substitutions and one or two indels in each read.

A typical operation on these reads is to take them and map them to a position in a template which might be an already known genome or transcriptome (a known set of sequences that occur in messenger RNA). Ideally this map would locate the read at exactly the position where it occurred in the particular organisms DNA or RNA. This may be used to detect differences between a particular individual and a standard genome or some other individual. In this case there may be small differences between the reads and the genome they are being matched against (these may be both substitutions or indels as with errors).

Existing tools take a very long time to do this because of the large number of reads, the size of the templates and the need to allow for differences between the reads and the template. For example, the tool called BLAST compares every read against every position in the template which takes time proportional to the product of the size of the template and the number of reads.

The applicant's prior application published as U.S. 2008/0256070 discloses a method of cataloguing a data structure by associating indexes of data items with position information, the disclosure of which is herein incorporated by reference.

It is an object of the invention to provide an improved method and system for analysing data sequences or to at least provide the public with a useful choice.

### SUMMARY OF THE INVENTION

According to a first aspect there is provided a method of generating an index for one or more data sequence including the steps of:
a. applying a mask to the data sequence at a plurality of locations;
b. extracting sequences of unmasked values of portions of the data sequence at each location to generate extracted values; and
c. creating index key values based on the extracted values.

The data sequence may be one or more reference templates and/or one or more sample sequences, such as DNA or RNA sequences.

Each index key value may be based upon a concatenated form of each extracted value, although other transformations may be employed.

A number of different masks may be applied to the data sequence at a number of locations. At least some of the masks may include indels and/or substitutions. The masks may be manually or computer generated.

If a new index value is the same as an existing index value then a sub-index key value may be created, which may be the portion of the data sequence (read) used to generate extracted values. The identity of the mask used to create an index may also be stored in association with an index value.

There is further provided a method of indexing a sample data sequence including the steps of:
a. applying a mask to reads of the sample data sequence to produce extracted sequences; and
b. storing an identifier for each read in association with a corresponding index key value produced by the above method.

For each identifier for each read a value may be stored corresponding to the read of the sample data from which the extracted sequence is derived and/or a value corresponding to the position of a corresponding sequence in a reference template and/or a value corresponding to the mask used to obtain the extracted sequence.

There is further provided a method of evaluating a sample data sequence in which read values are associated with index key values according to the above method, including the steps of:
a. comparing read values with corresponding portions of a reference template based on corresponding index key values; and
b. determining scores for the reads based on the comparison and associating the scores with the reads.

Reads may be rejected based upon the comparison. A read may be rejected if there is more than one position at which it has a best score. A read may be rejected if its score falls below a threshold score level.

There is also provided a sequencing system including:
a. a sequencing machine which analyses a biological sample and outputs a nucleotide sequence of the sample; and
b. a data sequence analyser which:
   i. receives reads from the sequencing machine;
   ii. applies masks to the reads and/or one or more reference sequence to form extracted sequences;
   iii. forms index key values based on the extracted sequences; and
   iv. stores read identifiers in association with a corresponding index key value.

The index key values may be based on masked read values and/or masked template values.

The system may include a mask generator to automatically generate masks. The masks may include indels and/or substitutions.

A single index may be used and the identity of the mask used to create each index key value may be stored in association with the index key value. Alternatively multiple indexes may be formed and each index key value may be based on the identity of the extracted sequence and the mask used to create the extracted sequence.

The system may include an evaluation engine which scores each read based on an evaluation of each read and a portion of a reference sequence having the same index key value as the read. A read may be rejected if it has the same best score for a threshold number of portions of the reference sequence. The threshold value may be 1 or more. A read may be rejected if the score is below a threshold value.

There is further provided a method of evaluating a sample data sequence including the steps of:
a. forming a database of read values of the data sequence by applying a set of masks to the reads and storing identifiers for each reads in association with index key values derived from the masked value of the read;
b. forming a database of read values of a template sequence by applying a set of masks to the reads and storing identifiers for each read in association with index key values derived from the masked value of the read;
c. comparing reads from the data sequence and template sequence having the same index key values and evaluating the reads based on the comparison.

### BRIEF DESCRIPTION OF THE DRAWINGS

The accompanying drawings which are incorporated in and constitute part of the specification, illustrate embodiments of the invention and, together with the general description of the invention given above, and the detailed description of embodiments given below, serve to explain the principles of the invention.

FIG. 1 shows a sequencing system.

FIG. 2 shows a process for creating an index based on reads.

FIG. 3 shows a process for analyzing a read set.

FIG. 4 shows a process for creating an index based on one or more reference template.

FIG. 5 shows a process for creating an index based on reads and one or more reference template.

FIG. 6 shows a block diagram of a data sequence analyser.

### DETAILED DESCRIPTION

The following description is given in relation to methods and systems suitable for matching sequences of nucleotides (RNA or DNA) quickly and with low memory use. It is to be appreciated that the techniques described may be applied as appropriate to other types of sequences.

Terminology

In this specification the following terms are used to describe sequences as follows:
The term “reads” is used to describe a number of subsequences obtained by sampling a sampled sequence. The reads will typically be obtained from multiple locations within the sampled sequence. The reads may be all of the same length; referred to as the “read length” (or R in the formulae below) or of different lengths when the mask includes indels.
The term “template” refers to a reference sequence which consists of one or more relatively long sequences (typically longer than the reads).

When two sequences are compared with each other we talk about a substitution when the nucleotides at the same position in the two sequences is different.

When two sequences are compared with each other we talk a about an insertion or deletion (indel) when the two sequences can be made to line up by deleting or inserting a nucleotide at a specified position in one of the two sequences.

A “mask” defines values at specified positions of a read that are to be retained or masked.

A mask set is a set of different masks specified for reads of a particular length (each mask must fit within the specified read length).

With a given mask there may also be one or more indel masks associated with it. Each indel mask is modified to help the process of matching an indel. This is done by inserting or deleting one or more positions at selected places in the original mask. Masks may be created manually by a user or be computer generated. Computer generated masks may be generated based on stored parameters or user prescribed parameters or a combination of these.

A mask set can be used in one of the following ways:
1. Given a mask it is possible to extract a subsequence from a read by selecting the nucleotides which lie at the positions specified in the mask. These nucleotides may then be concatenated (in the same order as they occurred in the read to form an extracted sequence).
2. Similarly a subsequence can be extracted by applying a mask at a specified position in the template.

Sequencing System

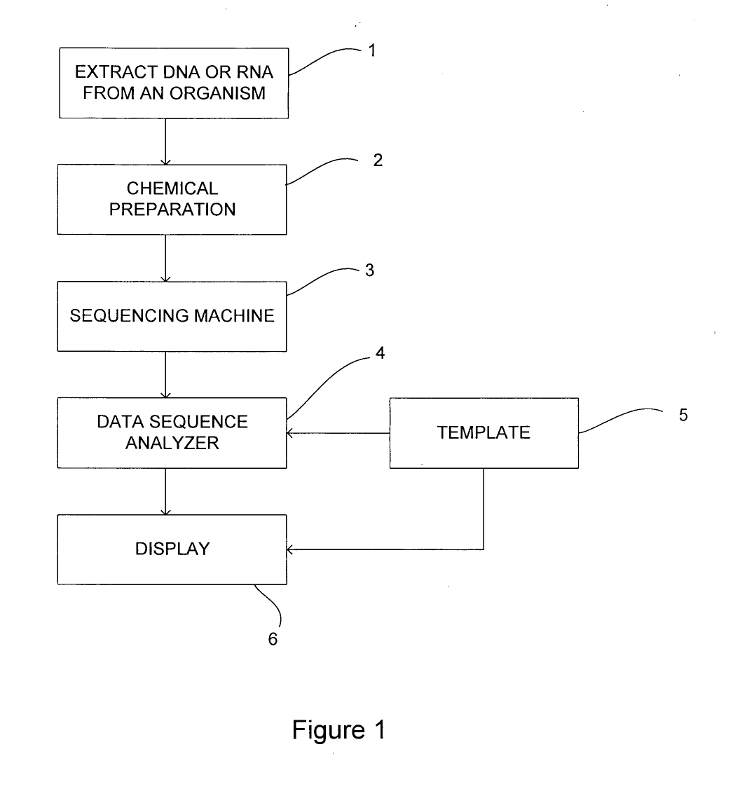

Referring to FIG. 1 a sequencing system for sequencing nucleotides such as DNA and RNA is shown. DNA or RNA is extracted from an organism in step 1, undergoes chemical preparation in step 2 and is then sequenced by sequencing machine 3. Sequencing machine 3 provides a sample sequence to data sequence analyser 4 for analysis. The sample sequence data may be of a specified read length or may be of a greater length (from which reads of the required length are obtained by moving a window of the required read length along the sample sequence data). Data analyser 4 may analyse the sample sequence with respect to one or more reference template 5 and supply the results of analysis to display 6 for viewing or to other equipment for further analysis.

The chemical preparation and sequencing processes may be performed as described in U.S. Pat. No. 5,750,341, U.S. 2006/0029957 A1 or “Sequence information can be obtained from single DNA molecules” by Ido Braslavsky, Benedict Hebert, Emil Kartalov and Stephen R. Quake PNAS Apr. 1, 2003 vol. 100 no. 7 3960-3964, the disclosure of which is herein incorporated by reference.

Methods for generating an index, analysing reads and preparing masks will now be described by way of example.

As in the applicant's prior application U.S. 2008/0256070 index values associated with reads are stored in association with position information. In this method, however, index key values are determined by applying a mask to read values and/or one or more template. Index values may be determined in at least the two following ways:
1. Multiple indexes—In this case a separate index may be created for each mask and index key values may be based on the value of each read and/or one or more template after a mask is applied and the result is concatenated or otherwise computed (e.g. zeros substituted for masked values).
2. Single index—each index value may be based upon the mask applied and the value of each read after the mask is applied and/or one or more template after a mask is applied and the result is concatenated or otherwise computed.

Such indexes can be arranged in many ways including by sorting the index key values or by sorting a hash of the index key values (see paragraphs 96 and 97 of U.S. 2008/0256070).

Build an Index Using the Reads

In this method the index for each mask is created “on the fly” using an extracted read sequence from each read for that mask as the index key value (i.e. the mask is applied to the read, the unmasked values are concatenated and a value derived from the concatenated sequence is used as an index key value for the read). Optionally an identifier for the mask; an identifier for the read (i.e. the read before masking); the position of the mask in the read; and/or an identifier for the mask may be associated with the index key value.

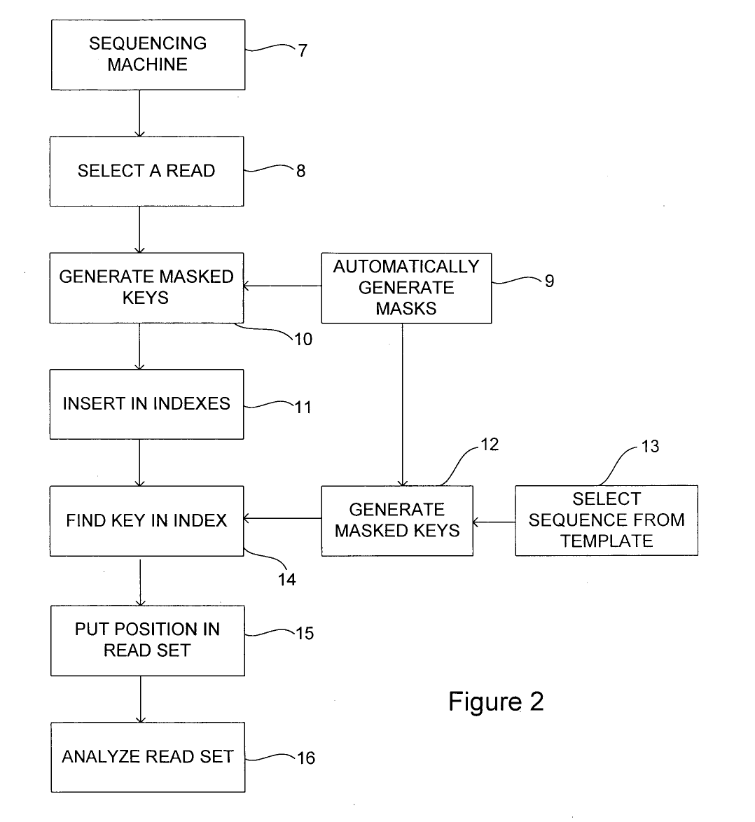

As shown in FIG. 2 sequencing machine 7 supplies a sample sequence, which may be of a read length or greater. A read is selected in step 8 and an automatically generated mask 9 is applied in step 10 to output an extracted read sequence. The extracted read sequence is concatenated in step 11 and a corresponding value is generated as an index key value for that read. The corresponding value may simply be a numeric equivalent of the concatenated sequence or a hash etc or an invertible transformation of the concatenated sequence. Multiple masks may be sequentially applied in step 10 to create index key values for each mask and extracted read sequence combination.

For each mask 9 and any associated indel masks extracted template sequences are determined in step 12 for each position in each template sequence 13. Typically this is done by computing the extracted sequences for each mask at one position then moving to the next position, but it could be done in other ways.

A corresponding value for each extracted sequence from a template 13 (determined in the same manner as for the reads) is looked up in the index generated from reads in step 11. If an identical match is found in the index then the associated value (the read identifier) is placed in a set associated with the position in the corresponding template called the position set in step 15.

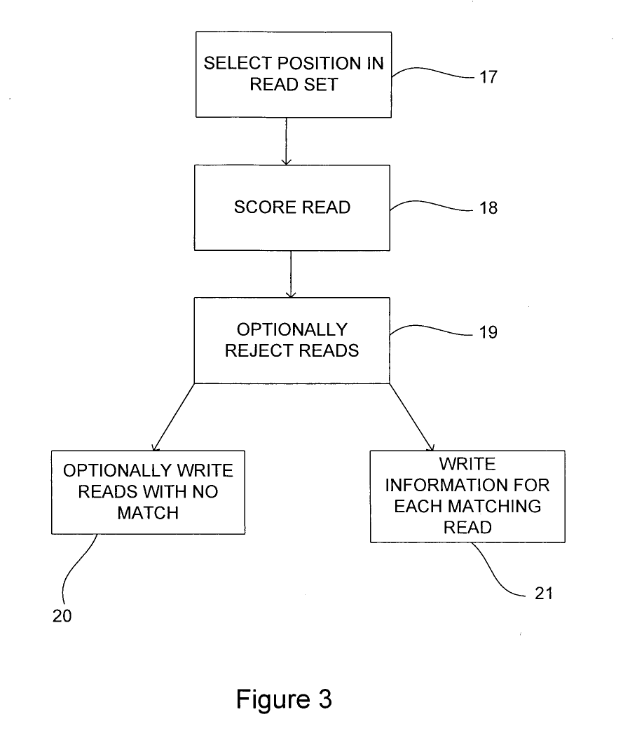

The reads may then be analysed in step 16 as shown in FIG. 3.

For each read in the position set 17 we may optionally:
1. compute a score which measures how well the complete read matches at that position in the template at step 18. (i.e. in Example 1 compare read 1 with template 7).
2. reject or include the read on the basis of its score in step 19.

We may then record for each read the position where it occurred in the template and optionally the score of the match in step 21 or optionally that there was no match in step 20.

Once all positions have been processed then for each read we may:
1. optionally reject reads where there is more than one position which has the equal best score; and/or
2. record the information for each read. Optionally this may include all the positions a read was matched at or only those with the best scores. An identifier for the read, as well as optionally the position it occurs in the template, as well as optionally the score for the match at that position may be recorded.

Optionally identifiers may be recorded for all reads whose best score occurred for more than one position in the template. Optionally that score may also be recorded.

Build an Index Using the Template

In this embodiment one or more indexes are formed based on one or more template sequence (a reference or target sequence). This can be done in a number of ways. For example, a single index can be created and when each extracted sequence is stored in it, the identity of the associated mask can be stored with it. Alternatively a separate index can be created for each of the masks in the mask set as in the previous example.

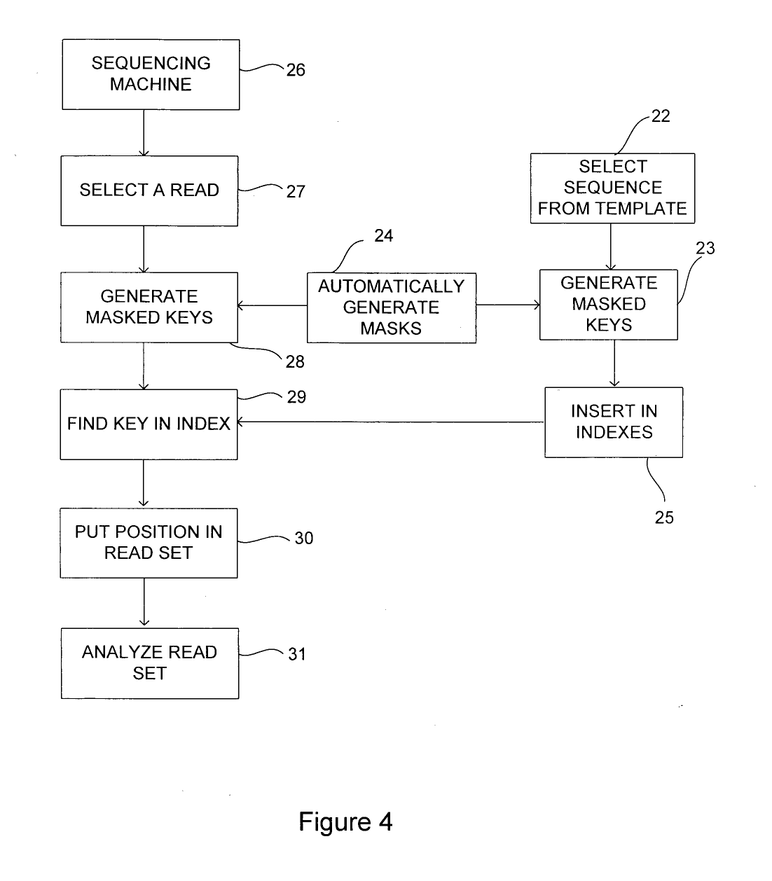

As shown in FIG. 4 sequences are generated from one or more reference templates in step 22. Typically a window of the read length is sequentially shifted along each reference template to produce all possible template sequences of the read length.

The template sequences output in step 22 are then masked in step 23 according to mask templates generated in step 24. Optionally there are other ways of accomplishing the same result more efficiently by recognizing that some masks are just versions of other masks shifted within the read length. For a set of masks which are all shifted versions of each other only one of these masks need be extracted at all positions within the template.

The masked extracted sequences from step 23 are then concatenated and used to generate index key values (typically a numerical value corresponding to the concatenated sequence although other conversion algorithms may be employed—e.g. instead of concatenating the sequences null values could be substituted for masked values). The generated index key values from step 23 are used to populate the index in step 25 for each mask. The position and identifier for the template sequence and optionally an identifier for the mask may also be associated with each index key value.

Reads may be processed as follows. Sequencing machine 26 generates sequences from which reads are selected in step 27 and masked in step 28 according to masks generated in step 24. Each read will be processed with each mask and any associated indel masks so that all possible masked output sequences are output from step 28. Once the index has been formed based on the one or more template sequences the output sequences from step 28 are concatenated and the corresponding value (determined as for the index key values) is looked up in the index created from the template sequences in step 29 to find a match.

If an identical match is found with an extracted sequence stored in the index then the associated value (the template sequence and its position) is placed in a set associated with the read in step 30 to create a “read set”. The read set may then be analysed as follows:

Take each read in the read set and optionally:
1. compute a score which measures how well the read matches at that position in the template; and/or
2. reject or include the read on the basis of its score

Once all positions have been processed then for each read optionally:
1. reject reads where there is more than one position which has the equal best score; and/or
2. record the information for each read. This may include all the positions the read was matched at or only those with the best scores. An identifier for the read may be recorded, as well as optionally the position it occurs in the template, as well as optionally the score for the match at that position.

Optionally the identifiers for all reads which had no match against any position in the template may be recorded.

Optionally the identifiers for all reads whose best score occurred for more than position in the template may also be recorded. Optionally that score may be recorded also.

Build an Index Using the Template and Build an Index Using the Reads

According to this embodiment indexes may be created based both on reads and one or more reference template. One or more indexes are created for each of the masks and for both the reads and the template. This can be done in a number of ways including the methods described above. For example, a single index can be created and when each extracted sequence is stored in it, the identity of the associated mask can be stored with it. Alternatively a separate index can be created for each of the masks in the mask set.

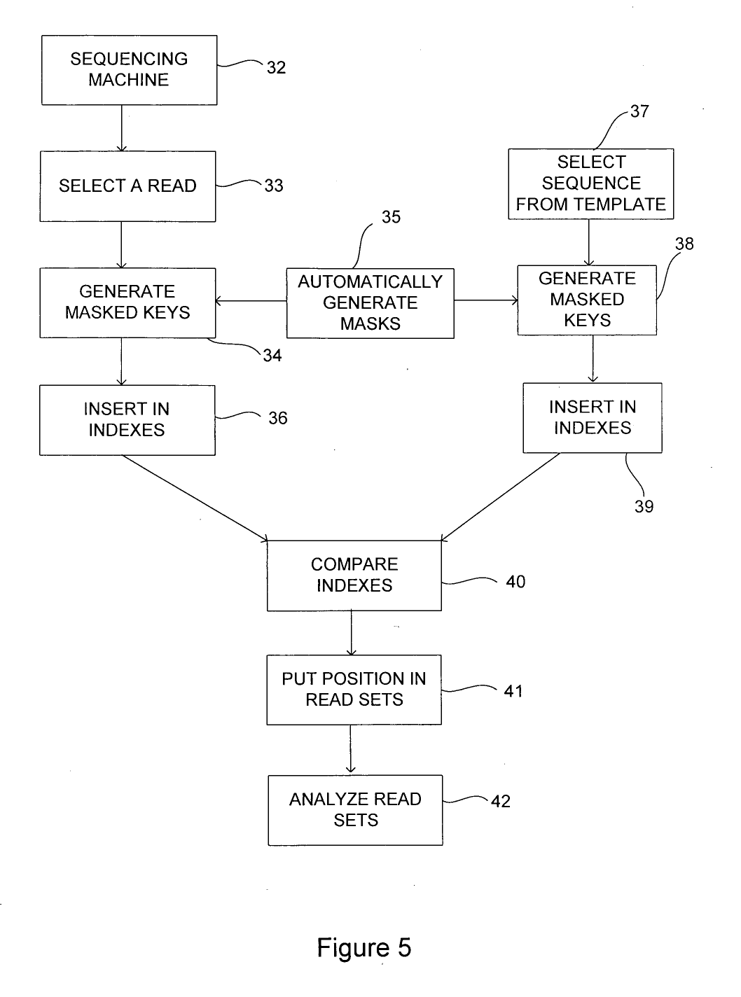

As shown in FIG. 5 reads are selected in step 33 from sequences supplied from sequencing machine 32. The reads are masked in step 34 according to masks generated in step 35. The extracted read sequences output from step 34 are concatenated and used to generate index key values associated with each read to form a “read index” in step 36. Optionally an identifier for the mask and an identifier for the read may be associated with each read index key value.

Likewise sequences of reference templates of the read length are sequentially selected in step 37 so that all possible sequences of read length are output. These sequences are then masked in step 38 using the mask combinations generated in step 35 and the extracted template sequences output from step 38 are concatenated and used to generate index key values to form a “template index”. Optionally an identifier for the mask and an identifier for the template sequence may be associated with each template index key value.

In step 40 corresponding pairs of read index and template index key values for each mask are compared. For many methods of indexing this can be done more efficiently than repeatedly searching an index. In particular it can reduce the number of accesses to RAM on computers with a cache for RAM. For each identical match found in the two indexes place the template sequence and position (stored in the template index) in a set associated with the read (stored in the read index) in step 41 and analyse the read sets in step 42 as follows.

Take each read in the read set and optionally:
1. compute a score which measures how well the read matches at that position in the template, and
2. reject or include the read on the basis of its score

Once all positions have been processed then for each read optionally:
1. reject reads where there is more than one position which has the equal best score; and/or
2. record the information for each read. Optionally include all the positions it was matched at or only those with the best scores. Record an identifier for the read, as well as optionally the position it occurs in the template, as well as optionally the score for the match at that position.

Optionally the identifiers for all reads which had no match against any position in the template may be recorded.

Optionally the identifiers for all reads whose best score occurred for more than position in the template may be recorded. Optionally that score may be included in the output.

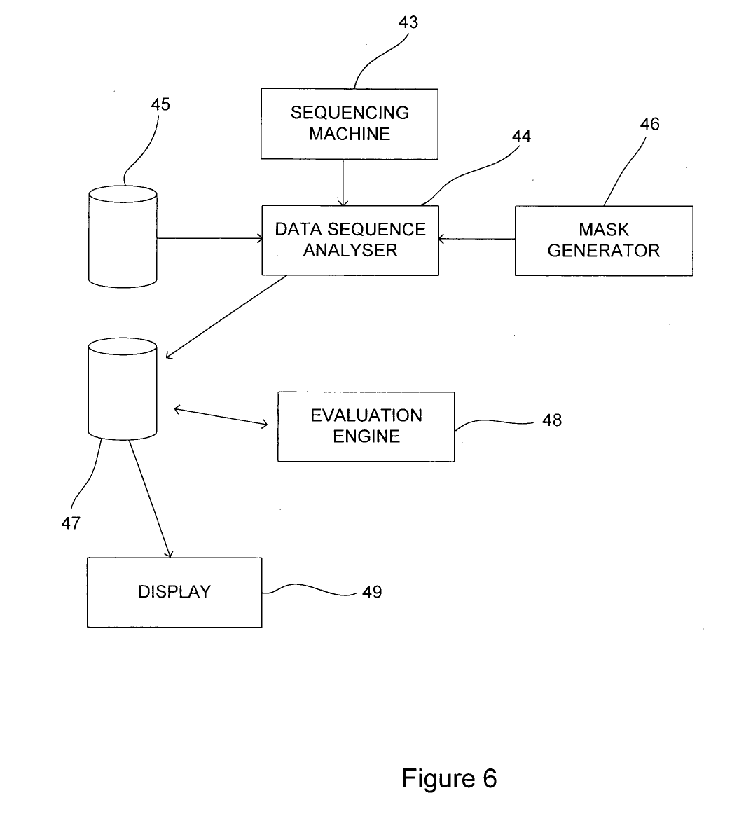

FIG. 6 shows a sequencing system for performing the method described above in which sequencing machine 43 supplies reads to data sequence analyser 44 which generates index key values based on mask reads from the sequencing machine and/or reads from template sequences in template database 45. The index key values generated are used in database 47 to store reads from the sequencing machine and/or the template sequences. Reads from the sequencing machine are compared with the template sequences by evaluation engine 48 to produce a score for reads (which may be accepted or rejected as described above). Reads and their score and other related information may be viewed on display 49 or otherwise provided for further use. Data sequence analyser 44, mask generator 46 and evaluation engine 48 may be specific circuits or one or more general purpose computer programmed to perform the method described.

### EXAMPLE 1

### Substitutions

The first example shows the process of extracting a sequence from a read and a corresponding template in the presence of two substitutions in the template sequence.

It starts at the top (line 1) with a single read of length 15. Associated with this is a single mask with 9 positions marked in the mask (indicated by an X) (see line 2). The mask is applied to the read and selects 9 nucleotides from the read (see line 3). These reads are then concatenated to form the extracted sequence (see line 4).

The template is given on line 7. It is at least as long as the read. At two positions there has been a substitution in the template, these are marked with an S and the use of a lower case letter to indicate the substituted nucleotide. Line 6 shows the same mask as on line 2. This is used to mask the template (line 5) and when these nucleotides are concatenated they lead to the same extracted sequence as that from the read (line 4).

The extracted sequence can be used to generate an index key value and thus to associate the position in the template with the read.

### EXAMPLE 2

### Substitution and Indel

The second example shows the process of extracting a sequence from a read and a corresponding template in the presence of one substitution and one indel (an insertion) in the template sequence.

Lines 1 through 4 are the same as Example 1.

The template is given on line 7. It is at least as long as the read. At one position there has been a substitution in the template, marked with an S and the use of a lower case letter to indicate the substituted nucleotide. At another position there has been an insertion in the template, marked with an I and the use of a lower case letter to indicate the inserted nucleotide. Line 6 shows an indel mask associated with the mask on line 2.

This is used to mask the template (line 5) and when these nucleotides are concatenated they lead to the same extracted sequence as that from the read (line 4).

The extracted sequence can be used to generate an index key value and thus to associate the position in the template with the read.

### EXAMPLE 3

### Mask Set

The first example of a mask set shows ten masks that together are able to correctly find all reads of length 15 with up to two substitutions. They may be able to correctly map with more substitutions but are not guaranteed to do so. Also they may be able to correctly map with some indels but are not guaranteed to do so.

### EXAMPLE 4

### Mask Set including Indel Masks

The second example of a mask set shows the ten masks from example 1 together with an additional 14 indel masks that together are able to correctly find all reads of length 15 with up to two substitutions and one indel. They may be able to correctly map with more substitutions but are not guaranteed to do so. Also they may be able to correctly map with more indels but are not guaranteed to do so.

Masks 1, 7 and 10 do not have any associated indel masks. Masks 2, 3, 4, 8, and 9 have two associated indel masks. Mask 5 has four associated indel masks.

Mask sets can be constructed by humans however they can also be constructed automatically. This reduces the burden on the human to come up with a suitable mask and also can be used to guarantee properties of the mask sets such as detecting specified numbers of indels and substitutions or of ensuring that only a certain fraction of reads will be missed given a probabilistic model of the rate at which indels and substitutions occur.

The following two algorithms are ways of automatically generating mask sets given the following parameters:
R the length of the reads
W the minimum number of unmasked positions in each mask
S the number of substitutions to be allowed

The process of adding indel masks will be addressed later.

Both algorithms work with the idea of a number of chunks in the mask. Each chunk is an adjacent set of positions. Each chunk will have all its positions included or not in any particular mask. The major part of both the algorithms is working out how many chunks are needed and how long they should be.

Mask Set Generation Algorithm 1

The algorithm uses a number of internal variables
k—the number of chunks used for unmasked portions of the mask
c—the total number of chunks in the entire mask
t—the number of positions in each chunk
w—the total number of unmasked positions (this must be ≧W)
r—the total number of positions used in the mask (this may be less than the actual read length R, any remaining positions in the read are effectively unused)

k is varied from 1 to W;
c=k+s
t=┌W/k┐
w=k*t
r=c*t
if w≧W and r≦R then the algorithm has found a solution

The mask set is then found by generating all different permutations of c chunks where S of them are unmasked and the remaining c−S are masked.

Mask Generation Algorithm 1.

| R = 17 | W = 9 | S = 2 | | | |
|---|---|---|---|---|---|
| k | c = k + S | t = ceil(W/k) | w = k * t | r = c * t | ok |
| 1 | 3 | 9 | 9 | 27 | X |
| 2 | 4 | 5 | 10 | 20 | X |
| 3 | 5 | 3 | 9 | 15 | ✓ |
| 4 | 6 | 3 | 12 | 18 | X |
| 5 | 7 | 2 | 10 | 20 | X |
| 6 | 8 | 2 | 12 | 16 | ✓ |
| 7 | 9 | 2 | 14 | 18 | X |
| 8 | 10 | 2 | 16 | 20 | X |
| 9 | 11 | 1 | 9 | 11 | ✓ |

The example above has a read length of 17 and a minimal unmasked length of 9 and two substitutions are to be allowed when detecting matches.

The table shows the calculations for each value of k from 1 to 9. There are three potential solutions at k=3, 6 and 9.

Examining the solution for k=3 further. The mask will have a total of 5 chunks (c), 3 (k) of these will be masked and each chunk will be three positions long (t). The diagram below shows a graphical representation of this

The top line shows the chunks and the fact that there is an overhang of two positions that are always masked out for read lengths of 17. When all permutations of the chunks are used to generate the masks then the result is the same 10 masks (ignoring the overhang) as in the earlier Example 1.

Note that any permutation of the positions leads to a new set of masks which can also detect mismatches with two substitutions.

Mask Set Generation Algorithm 2

The algorithm uses a number of internal variables
k—the number of chunks used for unmasked portions of the mask
c—the total number of chunks in the entire mask
t—the number of positions in the smallest chunks (some chunks may have length t+1)
a—the number of chunks which will have length t
b—the number of chunks which will have length t+1
w—the total number of unmasked positions for the mask with the smallest number of unmarked positions (this must be ≧W)

k is varied from 1 to R−S;
c=k+s
t=└R/k┘
w=k*t
b=R−t*c
a=c−b
w=if a≧k then k*t else k*t+k−a
if w≧W then the algorithm has found a solution

The mask set is then found by generating all different permutations of c chunks where S of them are unmasked and the remaining c−S are masked.

Mask Generation Algorithm 2.

| R = 17 | W = 9 | S = 2 | | | | |
|---|---|---|---|---|---|---|
| k | c = k + S | t = ceil(W/k) | b = R − t*c | a = c − b | w | ok |
| 1 | 3 | 5 | 2 | 1 | 5 | X |
| 2 | 4 | 4 | 1 | 3 | 8 | X |
| 3 | 5 | 3 | 2 | 3 | 9 | ✓ |
| 4 | 6 | 2 | 5 | 1 | 11 | ✓ |
| 5 | 7 | 2 | 3 | 4 | 11 | ✓ |
| 6 | 8 | 2 | 1 | 7 | 12 | ✓ |
| 7 | 9 | 1 | 8 | 1 | 13 | ✓ |
| 8 | 10 | 1 | 7 | 3 | 13 | ✓ |
| 9 | 11 | 1 | 6 | 5 | 13 | ✓ |
| 10 | 12 | 1 | 5 | 7 | 13 | ✓ |
| 11 | 13 | 1 | 4 | 9 | 13 | ✓ |
| 12 | 14 | 1 | 3 | 11 | 13 | ✓ |
| 13 | 15 | 1 | 2 | 13 | 13 | ✓ |
| 14 | 16 | 1 | 1 | 15 | 14 | ✓ |
| 15 | 17 | 1 | 0 | 17 | 15 | ✓ |

The example above has a read length of 17 and a minimal unmasked length of 9 and two substitutions are to be allowed when detecting matches.

The table shows the calculations for each value of k from 1 to 15. There are 12 potential solutions at k=3 to 15.

Examining the solution for k=3 further. The mask will have a total of 5 chunks (c), 3 (k) of these will be masked. Three (a) of these will be of length 3 (t) and two (b) will be of length 4 (t+1). The diagram below shows a graphical representation of this.

The top line shows the chunks and the fact that there are chunks of both sizes three and four. When all permutations of the chunks are used to generate the masks then the result is a similar set 10 masks as in the earlier Examples. Note that the number of unmasked positions varies from 9 (line 1) to 11 (lines 9 and 10).

Note that any permutation of the positions leads to a new set of masks which can also detect mismatches with two substitutions.

Indel Mask Generation Algorithm 3

Given a mask set which can detect mismatches of S substitutions then it is possible to automatically generate a set of indel masks associated with each mask in the mask set.

Consider the set of 10 masks in Example 3. Some of these have unmasked chunks flanked on both ends by masked chunks, these will be referred to as holes. Line 5 has two holes and lines 2 through 6 and 8 and 9 have just one hole. Lines 1, 7 and 9 have no holes.

Given the parameter I for the number of indels to be matched (as well as the S substitutions) the procedure for generating the indel masks from the individual masks is as follows:

Split the number I so that each hole in the mask is assigned a number from 0 to the minimum of I and the length of the hole (inclusive) and so that the sum of the numbers is less than or are equal to I. Each such assignment is a partition of I.

(Note that there is no partition if there are no holes.)

For example line 5 has two holes and if I is 2 then there are the following three partitions:

| | |
|---|---|
| 0 | 2 |
| 1 | 1 |
| 2 | 0 |

For each partition create a number of new indel masks as follows:

if a hole has been given a partition number i then
if i is zero do nothing
if i is greater than zero replace the hole with a new hole which has had either i positions deleted or i positions inserted.

Doing this systematically to all the holes gives a new set of masks. Indicating an insertion by + and a deletion by − the three partitions above give the following set of modifications to the holes (the diagram also shows the corresponding indel mask after applying the modification).

These methods have the following advantages:
they are very fast compared with other techniques;
they can allow for more differences between the read and the template than other techniques; and
they can be easily tuned to vary the amount of difference that is allowed between a read and the template.

Whilst the invention has been described, and has particular advantage, in relation to DNA/RNA sequencing it will be appreciated that the method may be applied to a range of suitable data sequences.

While the present invention has been illustrated by the description of the embodiments thereof, and while the embodiments have been described in detail, it is not the intention of the Applicant to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the spirit or scope of the Applicant's general inventive concept.

1. A method of generating an index for one or more data sequence including the steps of:
a. applying a mask to the data sequence at a plurality of locations;
b. extracting sequences of unmasked values of portions the data sequence at each location to generate extracted values; and
c. creating index key values based on the extracted values.

2. A method as claimed in claim 1 wherein the data sequence is one or more reference templates.

3. A method as claimed in claim 1 wherein the data sequence is one or more sample sequences.

4. A method as claimed in claim 3 wherein the sample sequences are DNA or RNA sequences.

5. A method as claimed in claim 1 wherein each index key value is based upon a concatenated form of each extracted value.

6. A method as claimed in claim 1 wherein a plurality of different masks are applied to the data sequence at a plurality of locations.

7. A method as claimed in claim 5 wherein at least some of the masks include indels.

8. A method as claimed in claim 5 wherein at least some of the masks include substitutions.

9. A method as claimed in claim 1 wherein at lease some of the masks are computer generated.

10. A method as claimed in claim 8 wherein masks are generated according to algorithm 1 as herein defined.

11. A method as claimed in claim 8 wherein masks are generated according to algorithm 2 as herein defined.

12. A method as claimed in claim 8 wherein masks are generated according to algorithm 3 as herein defined.

13. A method as claimed in claim 1 wherein if a new index value is the same as an existing index value then a sub-index key value is created.

14. A method as claimed in claim 13 wherein the sub-index key value is the portion of the data sequence (read) used to generate extracted values.

15. A method as claimed in claim 1 wherein the identity of the mask used to create an index value is stored in association with the index value.

16. A method as claimed in claim 1 wherein indexes are generated based on both reference templates and sample sequences.

17. A method of indexing a sample data sequence including the steps of:
a. applying a mask to reads of the sample data sequence to produce extracted sequences; and
b. storing an identifier for each read in association with a corresponding index key value produced by the method of claim 1.

18. A method as claimed in claim 17 wherein for each identifier for each read a value is stored corresponding to the read of the sample data from which the extracted sequence is derived.

19. A method as claimed in claim 17 wherein for each identifier for each read a value is stored corresponding to the position of a corresponding sequence in a reference template.

20. A method as claimed in claim 17 wherein for each identifier for each read a value is stored corresponding to the mask used to obtain the extracted sequence.

21. A method of evaluating a sample data sequence in which read values are associated with index key values according to the method of claim 17, including the steps of:
a. comparing read values with corresponding portions of a reference template based on corresponding index key values; and
b. determining scores for the reads based on the comparison and associating the scores with the reads.

22. A method as claimed in claim 22 wherein reads are rejected based upon the comparison.

23. A method as claimed in claim 23 wherein a read is rejected if there is more than one position at which it has a best score.

24. A method as claimed in claim 23 wherein a read is rejected if its score falls below a threshold score level.

25. A sequencing system including:
a. a sequencing machine which analyses a biological sample and outputs a nucleotide sequence of the sample; and
b. a data sequence analyser which:
i. receives reads from the sequencing machine;
ii. applies masks to the reads and/or one or more reference sequence to form extracted sequences;
iii. forms index key values based on the extracted sequences; and
iv. stores read identifiers in association with a corresponding index key value.

26. A system as claimed in claim 25 wherein the index key values are based on masked read values.

27. A system as claimed in claim 25 wherein the index key values are based on masked template values.

28. A system as claimed in claim 25 including a mask generator to automatically generate masks.

29. A system as claimed in claim 28 wherein the mask generator automatically generates masks including indels.

30. A system as claimed in claim 28 wherein the mask generator automatically generates masks including substitutions.

31. A system as claimed in claim 25 wherein a single index is formed and the identity of the mask used to create an index key value is stored in association with the index key value.

32. A system as claimed in claim 25 wherein multiple indexes are formed and each index key value is based on the identity of the extracted sequence and the mask used to create the extracted sequence.

33. A system as claimed in claim 25 including an evaluation engine which scores each read based on an evaluation of each read and a portion of a reference sequence having the same index key value as the read.

34. A system as claimed in claim 33 wherein a read is rejected if it has the same best score for a threshold number of portions of the reference sequence.

35. A system as claimed in claim 34 wherein the prescribed number is 1.

36. A system as claimed in claim 33 wherein a read is rejected if the score is below a threshold value.

37. A computer readable storage medium with computer executable instructions stored therein, said computer executable instructions being adapted to execute the method of claim 1.

38. A database formed by the method of claim 1.

39. A method of evaluating a sample data sequence including the steps of:
a. forming a database of read values of the data sequence by applying a set of masks to the reads and storing identifiers for each reads in association with index key values derived from the masked value of the read;
b. forming a database of read values of a template sequence by applying a set of masks to the reads and storing identifiers for each read in association with index key values derived from the masked value of the read;
c. comparing reads from the data sequence and template sequence having the same index key values and evaluating the reads based on the comparison.
